(19) United States
(12) Patent Application Publication
(10) Pub. No.: US 2005/0033230 A1
(43) Pub. Date: Feb. 10, 2005

(54) Prefillable intradermal delivery device with hidden needle and passive shielding

(76) Inventors: Paul G. Alchas, Wayne, NJ (US); Carlos E. Guillermo, San Luis Obispo, CA (US); David DeSalvo, Lake Hiawatha, NJ (US); Lucio Giambattista, East Hanover, NJ (US)

Correspondence Address: David W Highet;Becton Dickinson and Company, 1 Becton Drive, Franklin Lakes, NJ, 07417, US

(21) Appl. No.: 10/474,603

(22) PCT Filed: Feb. 15, 2002
(86) PCT No.: PCT/US02/04703

(51) Int. Cl. A61M 5/00

(52) U.S. Cl. 604/117

(57) ABSTRACT

An intradermal needle assembly (10) for injecting a substance into the skin of an animal is disclosed. A hub (16) is attachable to a container (12) storing the substance. A needle (18) is supported by the hub (16) and has a forward tip (20) extending away from the hub (16). A limiter (22) surrounds the needle (18) and extends away from the hub (16) toward the forward tip (20) of the needle (18). The limiter (22) includes a generally flat skin engaging surface (24) extending in a plane generally perpendicular to an axis of the needle (18) and is adapted to be received against the skin (26) to administer an intradermal injection. The limiter (22) is movable between a first position (28), wherein the skin engaging surface (24) shields the needle (18), and a second position (30), wherein the forward tip (20) extends beyond the skin (26) upon depressing the skin engaging surface (24) against the skin (26).

### FIELD OF THE INVENTION

The present invention relates generally to an intradermal injection assembly having a limiter positioned to limit the penetration of a forward tip of a needle cannula into the dermis of an animal. More particularly, the present invention relates to an intradermal delivery assembly having an integrated, passive shielding device to shield the forward tip of the needle cannula prior to and subsequent to administering the intradermal injection.

### BACKGROUND OF THE INVENTION

Intradermal injections are used for delivering a variety of substances. Many of these substances have proven to be more effectively absorbed into or react with the immune response system of the body when injected intradermally. An intradermal injection is made by delivering the substance into the epidermis and upper layer of the dermis. Below the dermis layer is subcutaneous tissue (also sometimes referred to as the hypodermis layer) and muscle tissue, in that order. There is considerable variation in the skin thickness both between individuals and within the same individual at different sites of the body. Generally, the outer skin layer epidermis has a thickness of between 50 to 200 microns, and the dermis, the inner and thicker layer of the skin, has a thickness between 1.5 and 3.5 mm. Therefore, a needle cannula that penetrates the skin deeper than about 3.0 mm has a potential of passing through the dermis layer of the skin making the injection into the subcutaneous region, which may result in an insufficient immune response, especially where the substance to be delivered intradermally has not been indicated for subcutaneous injection. Also, the needle cannula may penetrate the skin at too shallow a depth to deliver the substance and result in what is commonly known in the art as “wet injection” due to reflux of the substance from the injection site.

Due to the inherent limitations of the standard needle assembly, the standard procedure for making an intradermal injection is known to be difficult to perform, and therefore dependent upon experience and technique. This procedure is recommended to be performed by stretching the skin, orienting the needle bevel to face upwardly, and inserting a 26 gauge short bevel needle cannula to deliver a volume of 0.5 ml or less of the substance into the skin of the animal with the needle cannula being inserted into the skin at an angle varying from around 10 to 15° to form a blister or wheel in which the substance is deposited or otherwise contained.

The technique utilized to perform the standard intradermal injection is difficult and requires the attention of a trained nurse or medical doctor. Inserting the needle to a depth greater than about 3.0 mm typically results in a failed intradermal injection because the substance being expelled through the cannula will be injected into the subcutaneous tissue of the animal. As disclosed in pending U.S. patent application Ser. Nos. 09/834,438 and 09/417,671, an intradermal needle assembly has been developed for use with a prefillable container having a reservoir capable of storing a substance for injection into the skin of an animal. A hub portion, which is attachable to the prefillable container storing the substance, supports a needle cannula having a forward tip extending away from the hub portion. A limiter portion surrounds the needle cannula and extends away from the hub portion towards the forward tip of the needle cannula. The limiter includes a generally flat skin engaging surface extending in a plane generally perpendicular to an axis of the needle cannula and is adapted to be received against the skin of the animal to administer the intradermal injection of the substance. The needle forward tip extends beyond the skin engaging surface a distance of approximately 0.5 to 3.0 mm. Therefore, the limiter portion limits penetration of the needle into the dermis layer of the skin of the animal so that the vaccine is injected into the dermis layer of the animal.

The devices that have been adapted to administer an intradermal injection, and other devices utilizing needle cannulas with sharp tips to inject substances into the skin of the animal have typically included a fixed design where each of the components of the assembly are stationary relative to the other components. Therefore, the forward tip of the needle cannula remains exposed even after administration of the intradermal injection. With the advent of viral diseases transmitted through the contact of body fluid, it has become desirable to develop needle shielding devices that would prevent unintended access to the needle cannula subsequent to administering an injection. While these types of devices have been introduced to the marketplace for standard injection assemblies, they have not been developed in combination with an injection device adapted to administer an intradermal injection through the use of a limiter as described above.

In addition, it has become increasingly desirable to hide the needle prior to use to address any potential concerns by patients of seeing an exposed needle prior to use.

Therefore, it would be desirable to introduce an intradermal delivery assembly utilizing a limiter, as set forth above, to limit the penetration of a needle cannula into the dermis of an animal in combination with a shielding component to prevent unintended contact with the needle subsequent to administration of the intradermal injection, and to hide the needle prior to use.

### SUMMARY OF THE INVENTION AND ADVANTAGES

In contrast to the intradermal injection assemblies that have been disclosed in the past, it has been determined by the Applicant that it is feasible to include a shielding device with the intradermal assembly that can provide the benefit of shielding the forward tip of the needle cannula both prior to and subsequent to administering an intradermal injection.

The intradermal needle assembly of the present invention is for use with a prefillable container that includes a reservoir capable of storing a substance for injection into the skin of an animal. A hub portion is attachable to the prefillable container storing the substance and supports a needle cannula. The needle cannula includes a forward tip extending away from the hub portion. A limiter surrounds the needle cannula and extends away from the hub portion towards the forward tip of the needle cannula. The limiter includes a generally flat skin engaging surface extending in a plane generally perpendicular to an axis of the needle cannula and is adapted to be received against the skin of the animal to administer an intradermal injection of the substance.

The limiter is moveable between at least a first position where the skin engaging surface shields the forward tip of the needle cannula, and a second position where the forward tip extends beyond the skin engaging surface a distance sufficient to administer the substance into the dermis layer of the animal upon depression of the skin engaging surface against the skin of the animal. The limiter is biased toward the first position by a spring means that is compressible upon depressing the skin engaging surface against the skin of the animal. Therefore, after administering the intradermal injection and withdrawing the skin engaging surface from the skin of the animal, the limiter returns and locks to the first position to shield the forward tip of the needle cannula.

Alternatively, the limiter is circumscribed by a shield that is biased toward the forward tip of the needle cannula by the spring means. In this embodiment, the shield is moveable between the first position where the forward tip of the needle cannula is shielded, and the second position where the forward tip of the needle cannula extends beyond the skin engaging surface a distance sufficient to administer the substance into the dermis layer of the animal upon depression of the skin engaging surface against the skin of the animal. Accordingly, the limiter is fixed relative to the needle cannula and the hub portion and only the shield is moveable between the first and second positions.

The embodiments of the subject invention set forth above provide the benefit of administering an intradermal injection utilizing a depth limiting device that limits the penetration of the forward tip of the needle cannula into the dermis layer of the animal simplifying the method of administering the intradermal injection. Further, a passive shielding device is provided which shields the forward tip of the needle cannula both prior to and subsequent to administering the intradermal injection. Thus, the intradermal needle assembly prevents exposure to the forward tip of the needle cannula prior to administering the injection maintaining sterilization, and subsequent to administering the injection reducing the potential for a biohazard resulting from contact with the needle cannula.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is cross-sectional view of a first embodiment of the present invention showing a needle limiter capable of shielding a needle cannula while located in a first position;

FIG. 2 is cross-sectional view of the first embodiment of the present invention showing a needle limiter limiting the injection depth of the needle cannula while located in a second position;

FIG. 3 is cross-sectional view of the first embodiment of the present invention showing a needle limiter shielding a needle cannula while locked in the first position subsequent to administering the intradermal injection;

FIG. 4 is an exploded view of the first embodiment of the present invention;

FIGS. 5 and 6 show an alternate embodiment of the present invention in the first and second positions;

FIG. 7 shows the alternate embodiment locked in the first position subsequent to administering the intradermal injection;

FIG. 8 shows a further embodiment of the present invention with a sleeve capable of shielding the needle cannula while in a first position;

FIG. 9 shows the further embodiment with the sleeve located in a retracted position while administering the intradermal injection;

FIG. 10 shows the further embodiment locked in the first position subsequent to administering the intradermal injection;

FIGS. 11 and 12 show a still further embodiment of the present invention having a limiter slidably attached to a hub and being capable of shielding the needle cannula prior to and subsequent to administering the intradermal injection; and

FIG. 13 shows the still further embodiment locked in the first position subsequent to administering the intradermal injection.

### DETAILED DESCRIPTION OF THE PREFERRED EMBODIMENT

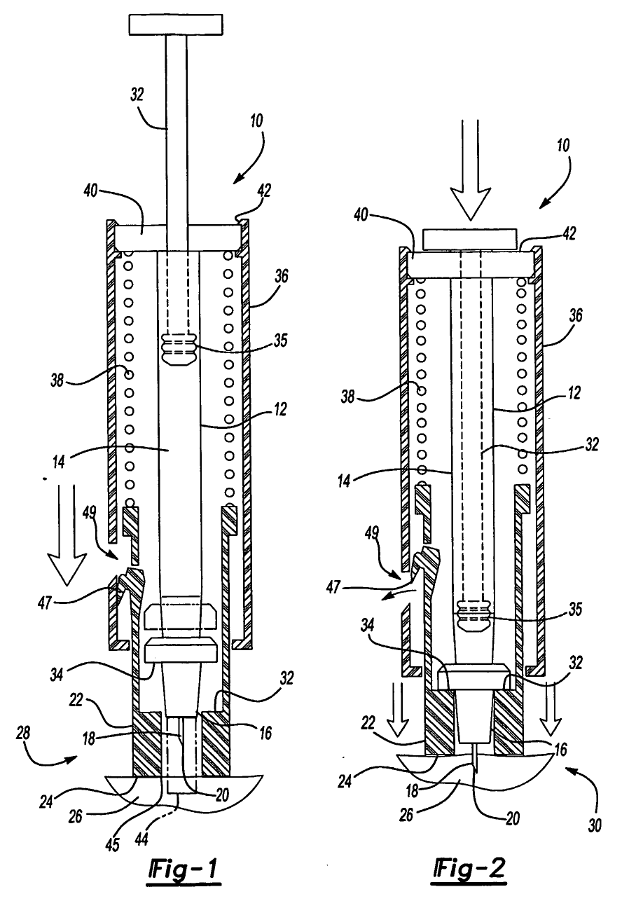

An intradermal needle assembly of the subject invention is generally shown at 10 of FIGS. 1, 2 and 3. The assembly includes a prefillable container 12 having a reservoir 14 capable of storing a substance for injection into the skin of an animal. A hub portion 16 is attachable to the prefillable container 12 and supports a needle cannula 18 having a forward tip 20 extending away from the hub portion 16.

A limiter 22 surrounds the needle cannula and extends away from the hub portion 16 toward the forward tip 20 of the needle cannula 18. The limiter 22 includes a generally flat skin engaging surface 24 extending in a plane generally perpendicular to an axis of the needle cannula 18. The skin engaging surface 24 is adapted to be received against the skin 26 of an animal to administer an intradermal injection of the substance.

The limiter 22 is moveable between a first position 28 as shown in FIG. 1 and a second position 30 as shown in FIG. 2. When the limiter is located in the first position 28, the skin engaging surface 24 shields the forward tip 20 of the needle cannula 18. When the limiter 22 is located in the second position 30, the forward tip 20 of the needle cannula 18 extends beyond the skin engaging surface 24 a distance sufficient to administer the substance disposed in the reservoir 14 into the dermis layer 26 of the animal. The assembly 10 includes a plunger 32 having a stopper 35 slideably disposed within the reservoir 14 of the prefillable container 12. As known to those of skill in the art, by depressing the plunger 32, the stopper 35 slides inwardly of the reservoir 14 causing the substance disposed in the reservoir 14 to be expelled from the prefillable container 12 through the needle cannula 18 into the skin 26 of the animal.

When the limiter 22 is disposed in the second position 30, the forward tip 20 of the needle cannula 18 extends beyond the skin engaging surface 24 a distance of approximately 0.5 to 3.0 mm. Therefore, the penetration of the needle cannula 18 into the dermis layer of the skin 26 is limited so that the substance is injected into the dermis layer 26 of the animal. The limiter 22 includes at least one stop 32 that is engageable with a counter stop 34 disposed upon the hub portion 16 to terminate movement of the limiter 22 rearwardly of the forward tip 20, thereby allowing only 0.5 to 3.0 mm of the needle cannula 18 to be exposed.

The limiter 22 is slideably disposed within a sleeve 36. Accordingly, the limiter 22 is concentrically aligned between the sleeve 36 and the prefillable container 12. The limiter 22 is biased outwardly of the sleeve 36 by a spring means 38. The spring means is represented in the figures as a coil spring, however, it should be understood by those of skill in the art that any resilient member may be used to bias the limiter 22 outwardly of the sleeve 36.

The spring means 38 is compressed between the limiter 22 and a rim 40 of the prefillable container 12 disposed upon an end of the prefillable container 12 opposite the limiter 22. The rim 40 of the prefillable container 12 is fixedly attached to the sleeve 36 by a plurality of snaps 42. A plug 44 is engageable with the needle cannula 18 to seal the prefillable container 12 at the needle cannula 18. The plug 44 is inserted through a central aperture 45 in the skin engaging surface 24 that is axially aligned with the needle cannula 18. The plug 44 is removed prior to administering the intradermal injection.

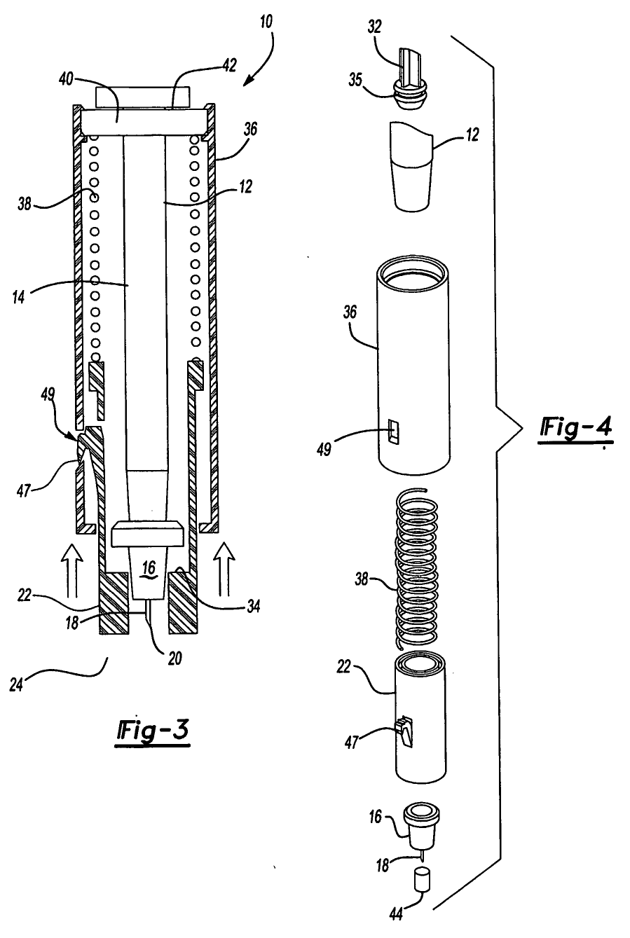

A least one catch 47 is disposed upon a side of the limiter 22 and is engageable with a slot 49 disposed in a side of the sleeve 36. The catch 47 is biased to engage the slot 49 when the limiter 22 returns to the first position 28 subsequent to administering the injection to prevent the forward tip 20 from being re-exposed. This is best represented in FIG. 3.

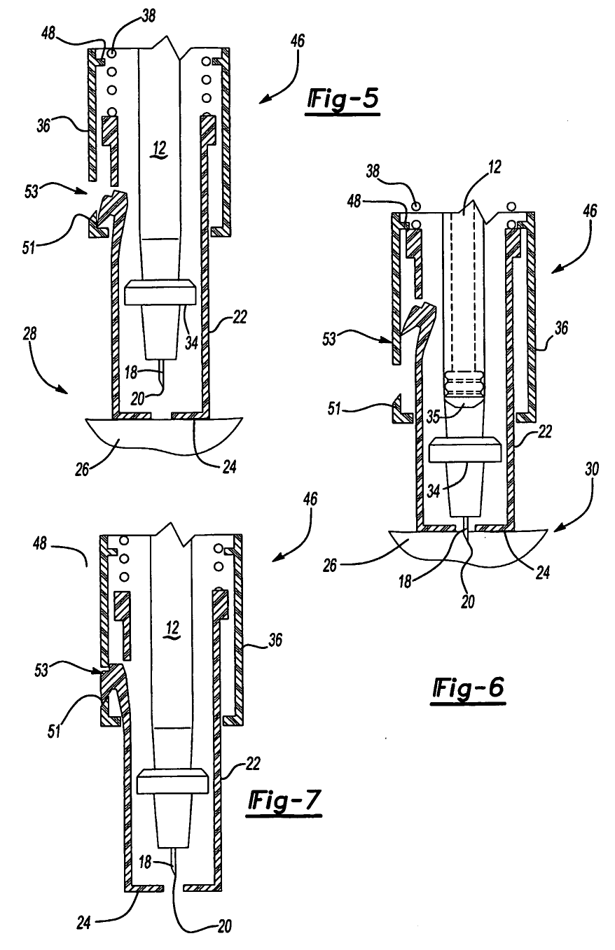

FIGS. 5, 6 and 7 show an alternate embodiment of the subject assembly generally at 46. At least one sleeve stop 48 is positioned rearwardly of the limiter 22 inside the sleeve 36. The sleeve stop 48 is spaced from the limiter 22 when the limiter is located in the first position 28. Movement of the limiter by depressing the skin engaging surface 24 against the skin 26 of the animal is terminated when the limiter 22 abuts the sleeve stop 48 locating the limiter 22 in the second position 30. As set forth above, when the limiter 22 is located in the second position 30, the forward tip 20 of the needle cannula 18 extends beyond the skin engaging surface 24 a distance between 0.5 and 3.0 mm. Upon withdrawing the alternate embodiment 46 from the skin 26 of the animal, the limiter 22 returns to the first position 28 shielding the needle cannula 18.

A least one catch 51 is disposed upon a side of the limiter 22 and is engageable with a slot 53 disposed in a side of the sleeve 36. The catch 51 is biased to engage the slot 53 when the limiter 22 returns to the first position 28 subsequent to administering the injection to prevent the forward tip 20 from being re-exposed. This is best represented in FIG. 7.

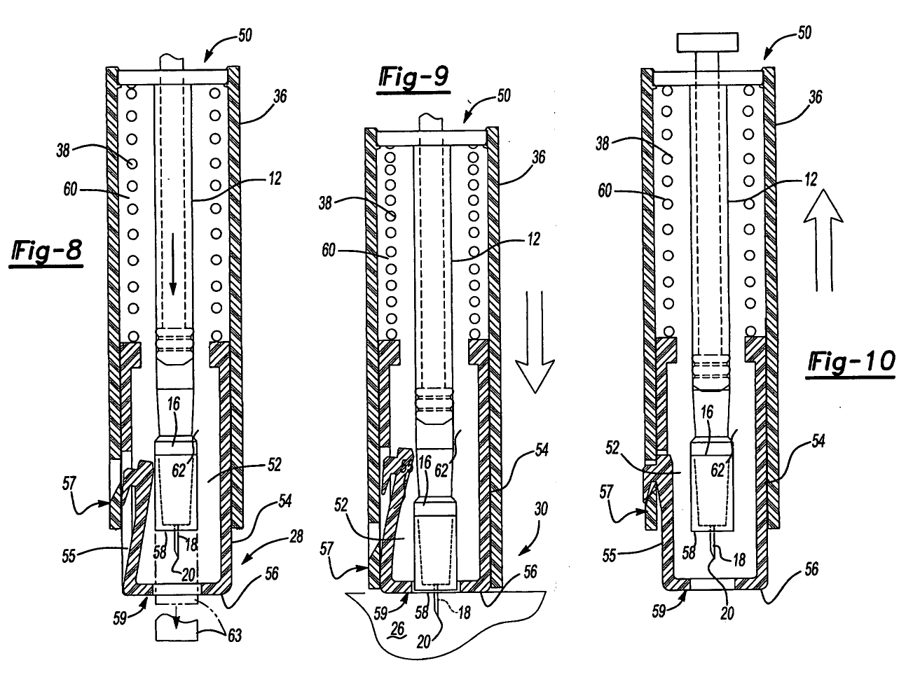

FIGS. 8 and 9 show a further alternate embodiment of the present invention generally at 50. A limiter portion 52 is affixed to the hub portion 16 in a stationary manner. A shield 54 circumscribes the limiter 52 and is concentrically aligned between the sleeve 36 and the prefillable container 12.

The shield 54 is slideable between the first position 28 and the second position 30. The spring means 38 biases the shield 54 towards the first position 28. The shield 54 shields the forward tip 20 of the needle cannula 18 when in the first position 28. The shield includes a skin engaging surface 56 that aligns with a generally flat skin engaging surface 58 of the stationary limiter portion 52 when the shield 54 is located in the second position 30. Upon depressing the limiter skin engaging surface 58 against the skin 26 of an animal, the shield 54 moves inwardly of the sleeve 36 exposing 0.5 to 3.0 mm of the needle cannula enabling the intradermal injection to be made into the skin 26 of the animal. Upon withdrawing the assembly 50 from the skin 26 of the animal, the shield 54 moves outwardly of the sleeve 36 returning to the first position 28.

An abutting member 62 abuts the hub portion 16 preventing the shield 54 from moving beyond the first position 28 or being ejected from the assembly 50 by the spring means 38.

A least one catch 55 is disposed upon a side of the limiter 22 and is engageable with a slot 57 disposed in a side of the sleeve 36. The catch 55 is biased to engage the slot 57 when the limiter 22 returns to the first position 28 subsequent to administering the injection to prevent the forward tip 20 from being re-exposed. This is best represented in FIG. 10.

A plug 63 is engageable with the needle cannula 18 to seal the prefillable container 12 at the needle cannula 18. The plug 63 is inserted through a central aperture 59 in the skin engaging surface 56 that is axially aligned with the needle cannula 18. The plug 63 is removed prior to administering the intradermal injection.

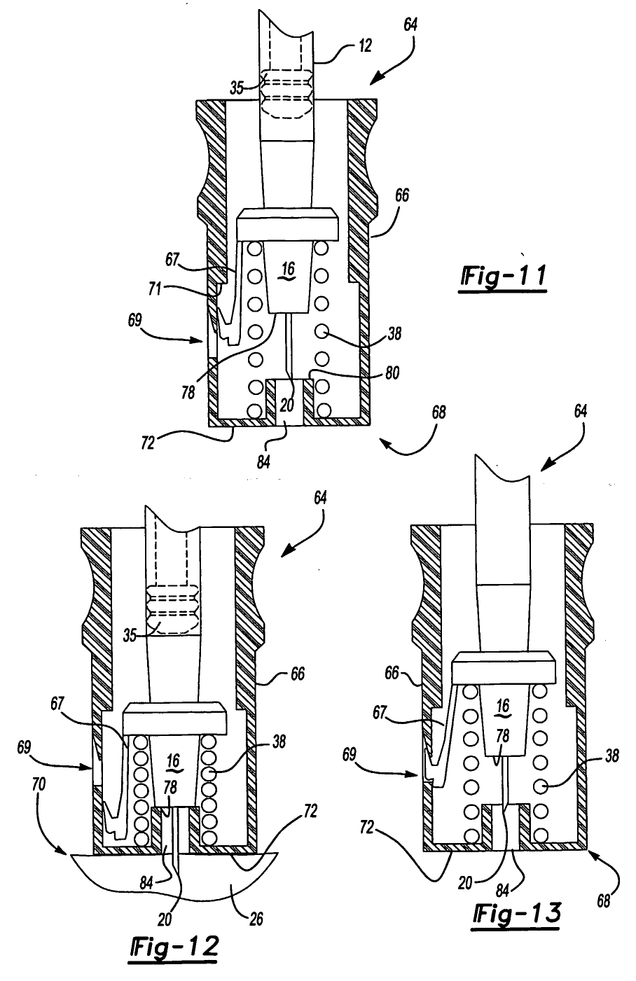

An additional alternate embodiment is generally shown at 64 in FIGS. 11 through 13. An alternate limiter 66 circumscribes the hub portion 16 and slideably engages the prefillable container 12.

The alternate limiter 66 is moveable between a first position 68 and a second position 70 relative to the hub portion 16. A skin engaging surface 72 as disposed upon the limiter 66 is engageable with the skin of the animal to administer the intradermal injection. The spring means 38 is compressed between the hub portion 16 and a rear side of the skin engaging surface 72. The spring means 38 biases the alternate limiter 66 to the first position 68. Upon depression of the skin engaging surface 72 against the skin 26 of the animal, the spring means 38 is compressed allowing the alternate limiter 66 to move from the first position 68 to the second position 70.

The hub portion 16 includes at least one catch 67 extending towards the forward tip 20 of the needle cannula 16. The catch 67 engages a slot 69 disposed in a wall of the limiter 66 to prevent the limiter 66 from being moved to second position 70 from the first position subsequent to administering the injection. A plurality of catches 67 may also be used to improve the stability of the limiter 66. A stop 71 is disposed upon an inner wall of the limiter and is engageable with the catch 67 to prevent the limiter 66 from being ejected from the assembly 64 by the spring means 38.

The forward tip 20 of the needle cannula 18 extends beyond the skin engaging surface 72 a distance of 0.5 to 3.0 mm when the alternate limiter 66 is disposed in the second position 70.

As shown in FIGS. 10 and 11, the alternate limiter 66 includes a stop 74 disposed upon an inner surface and is engageable with a rear member 76 of the hub portion 16. The stop 74 prevents the alternate limiter 66 from moving beyond the second position 70. Alternatively, as shown in FIGS. 12 and 13, a leading edge 78 of the hub portion 16 engages a flange 80 directed rearwardly from the skin engaging surface 72 to prevent the limiter portion 66 from moving beyond the second position. A plug 82 is engageable with the needle cannula 18 to seal the prefillable container 12 at the needle cannula. The plug 82 is inserted through a central aperture 84 in the skin engaging surface 72 that is axially aligned with the needle cannula 18. The plug 82 is removed prior to administering the intradermal injection.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, wherein reference numerals are merely for convenience and are not to be in any way limiting, the invention may be practiced otherwise than as specifically described.

1. An intradermal needle assembly for use with a prefillable container having a reservoir capable of storing a substance for injection into the skin of an animal comprising:
a hub portion being attachable to the prefillable container;
a needle cannula supported by said hub portion and having a forward tip extending away from said hub portion; and
a limiter surrounding said needle cannula and extending away from said hub portion toward said forward tip of said needle cannula, said limiter including a generally flat skin engaging surface extending in a plane generally perpendicular to an axis of said needle cannula and adapted to be received against the skin of the animal to administer an intradermal injection of the substance;
said limiter being movable between at least a first position, wherein said skin engaging surface shields said forward tip of said needle cannula, and a second position, wherein said forward tip extends beyond said skin engaging surface a distance sufficient to administer the substance into the dermis layer of the animal upon depression of said skin engaging surface against the skin of the animal.

2. An assembly as set forth in claim 1, further comprising spring means for biasing said limiter towards said first position.

3. An assembly as set forth in claim 1, wherein said forward tip of said needle cannula extends beyond said skin engaging surface a distance ranging from approximately 0.5 mm to 3.0 mm when said limiter is located in said second position thereby limiting penetration of said needle into the dermis layer of skin of the animal so that the substance is injected into the dermis layer of the animal.

4. An assembly as set forth in claim 3, further comprising a sleeve having the prefillable container fixedly disposed therein with said limiter slideably protruding therefrom.

5. An assembly as set forth in claim 4, wherein said sleeve includes a stop positioned to prevent said limiter from sliding beyond said second position.

6. An assembly as set forth in claim 1, further comprising catch engageable to prevent said limiter from moving from said first position to said second position subsequent to administering the intradermal injection.

7. An assembly as set forth in claim 1, wherein the prefillable container includes a rim disposed on an end opposite said hub and said spring means is provided between said rim and said limiter.

8. An assembly as set forth in claim 2, wherein said spring means is provided between said hub and said limiter thereby biasing said limiter towards said first position.

9. An assembly as set forth in claim 8, further comprising a stop positioned to prevent said limiter from sliding beyond said second position.

10. An assembly as set forth in claim 9, wherein said hub includes a catch engageable with said limiter when said limiter is returned to said first position thereby preventing said limiter from moving from said first position to said second position subsequent to administering the intradermal injection.

11. An assembly as set forth in claim 1, further comprising a plug comprising a resilient material applied over said forward tip of said needle cannula thereby sealing said prefillable container at said needle cannula.

12. An intradermal needle assembly for use with a prefillable container having a reservoir capable of storing a substance for injection into the skin of an animal comprising:
a hub portion being attachable to the prefillable container;
a needle cannula supported by said hub portion and having a forward tip extending away from said hub portion;
a limiter portion surrounding said needle cannula and extending away from said hub portion toward said forward tip of said needle cannula, said limiter including a generally flat skin engaging surface extending in a plane generally perpendicular to an axis of said needle cannula and adapted to be received against the skin of the animal to administer an intradermal injection of the substance, said needle forward tip extending beyond said skin engaging surface a distance sufficient to enable injection of the substance into the dermis layer of the animal; and
a shield circumscribing said limiter and being biased towards said forward tip of said needle cannula with at least a first position wherein said shield shields said forward tip, and a second position wherein said shield exposes said forward tip.

13. An assembly as set forth in claim 12, wherein said needle cannula extends beyond said skin engaging surface a distance ranging from approximately 0.5 mm to 3.0 mm.

14. An assembly as set forth in claim 13, further comprising a sleeve having the prefillable container fixedly disposed therein with said shield slideably protruding therefrom.

15. An assembly as set forth in claim 12, further comprising a spring means disposed within said sleeve and biasing said shield towards said forward tip of said needle cannula.

16. An assembly as set forth in claim 15, wherein the prefillable container includes a rim disposed on an end opposite from said hub whereby said spring means is compressed between said rim and said shield.

17. An assembly as set forth in claim 12, wherein said shield is moveable between said first position and said second position by depressing said skin engaging surface against the skin of the animal.

18. An assembly as set forth in claim 12, wherein said sleeve includes a stop positioned to prevent said limiter from sliding beyond said second position.

19. An assembly as set forth in claim 18, further comprising a catch engageable when said shield is returned to said first position thereby preventing said shield from moving from said first position to said second position subsequent to administering the intradermal injection.

20. An assembly as set forth in claim 12, further comprising a plug comprising a resilient material applied over said forward tip of said needle cannula thereby sealing said prefillable container at said needle cannula.

21. A method of administering an intradermal injection into the skin of an animal comprising the steps of:
providing a drug delivery device including a needle cannula having a forward needle tip, said needle cannula being in fluid communication with a substance contained in said drug delivery device;
shielding said forward tip of said needle cannula with a shielding surface by locating said shielding surface in a first position;
depressing said shielding surface against the skin of an animal thereby moving said shielding surface to a second position and exposing said forward tip of said needle cannula a length sufficient to administer an intradermal injection;
inserting said forward tip into the skin of the animal and engaging the surface of the skin with said skin engaging shielding surface such that said shielding surface limits penetration of the forward tip into the dermis layer of the skin of the animal;
expelling the substance from said drug delivery device through said forward tip into the dermis of the animal; and
retracting said shielding surface from the skin of the animal thereby allowing said shielding surface to move from said second position to said first position thereby shielding said forward tip of said needle cannula.

22. A method as set forth in claim 21 wherein said step of exposing said forward tip of said needle cannula is further defined as exposing a length of said needle cannula equal to approximately 0.5 mm to approximately 3.0 mm.

23. A method as set forth in claim 21 wherein said shielding surface is further defined as having a fixed angle of orientation relative to said needle cannula.

24. A method as set forth in claim 21 wherein said shielding surface is further defined as being adapted to be received against the skin of an animal.

25. A method as set forth in claim 21 further including the step of securing said shielding surface in said first position subsequent to expelling the substance from said drug delivery device into the dermis of the animal to prevent said forward tip of said needle cannula from being re-exposed.

26. A method as set forth in claim 21 further including the step of biasing said shielding surface towards said first position.

27. A method as set forth in claim 21 further including the step of providing a plunger being slideably disposed with said prefillable container and being depressible for expelling the substance from the container.

28. A method as set forth in claim 21 further including the step of unsealing said needle cannula prior to depressing said shielding surface against the skin of the animal.

29. An intradermal needle assembly for a prefillable container having a reservoir capable of storing a substance for injection into the skin of an animal comprising:
a hub portion provided on the prefillable container;
a needle cannula supported by said hub portion and having a forward tip extending away from said hub portion; and
a limiter surrounding said needle cannula and extending away from said hub portion toward said forward tip of said needle cannula, said limiter including a generally flat skin engaging surface extending in a plane generally perpendicular to an axis of said needle cannula, said limiter being movable between a first position in which said forward tip of said needle cannula is contained within said limiter, and a second position in which said forward tip of said needle cannula extends beyond said skin engaging surface a distance sufficient to administer the substance into the dermis layer of the animal upon depression of said skin engaging surface against the skin of the animal.

30. An assembly as set forth in claim 29, further comprising spring means for biasing said limiter toward said first position.

31. An assembly as set forth in claim 29, wherein said forward tip of said needle cannula extends beyond said skin engaging surface a distance ranging from approximately 0.5 mm to 3.0 mm when said limiter is located in said second position thereby limiting penetration of said needle into the dermis layer of skin of the animal so that the substance is injected into the dermis layer of the animal.

32. An assembly as set forth in claim 31, further comprising a sleeve having the prefillable container fixedly disposed therein with said limiter slideably protruding therefrom.

33. An assembly as set forth in claim 32, wherein said sleeve includes a stop positioned to prevent said limiter from sliding beyond said second position.

34. An assembly as set forth in claim 29, further comprising a catch engageable to prevent movement of said limiter from said first position to said second position subsequent to administering the intradermal injection.

35. An assembly as set forth in claim 29, wherein the prefillable container includes a rim disposed on an end opposite said hub and wherein said spring means is provided between said rim and said limiter.

36. An assembly as set forth in claim 30, wherein said spring means is provided between said hub and said limiter thereby biasing said limiter towards said first position.

37. An assembly as set forth in claim 36, further comprising a stop positioned to prevent said limiter from moving beyond said second position.

38. An assembly as set forth in claim 37, wherein said hub includes a catch engageable with said limiter when said limiter is returned to said first position thereby preventing said limiter from moving from said first position to said second position subsequent to administering the intradermal injection.

39. An assembly as set forth in claim 29, further comprising a plug comprising a resilient material applied over said forward tip of said needle cannula thereby sealing said prefillable container at said needle cannula.

40. An intradermal needle assembly for a prefillable container having a reservoir capable of storing a substance for injection into the skin of an animal comprising:
a hub portion provided on the prefillable container;
a needle cannula supported by said hub portion and having a forward tip extending away from said hub portion;
a limiter portion surrounding said needle cannula and extending away from said hub portion toward said forward tip of said needle cannula, said limiter including a generally flat skin engaging surface extending in a plane generally perpendicular to an axis of said needle cannula, said needle forward tip extending beyond said skin engaging surface a distance sufficient to enable injection of the substance into the dermis layer of the animal; and
a shield circumscribing said limiter and being movable between a first position, in which said forward tip of said needle cannula is shielded by said shield, and a second position, in which said forward tip of said needle cannula is exposed, said shield being biased towards said a first position.

41. An assembly as set forth in claim 40, wherein said needle cannula extends beyond said skin engaging surface a distance ranging from approximately 0.5 mm to 3.0 mm.

42. An assembly as set forth in claim 41, further comprising a sleeve having the prefillable container fixedly disposed therein with said shield slideably protruding therefrom.

43. An assembly as set forth in claim 40, further comprising a spring means disposed within said sleeve and biasing said shield towards said forward tip of said needle cannula.

44. An assembly as set forth in claim 43, wherein the prefillable container includes a rim disposed on an end opposite from said hub whereby said spring means is compressed between said rim and said shield.

45. An assembly as set forth in claim 40, wherein said shield is moveable between said first position and said second position by depressing said skin engaging surface against the skin of the animal.

46. An assembly as set forth in claim 40, wherein said sleeve includes a stop positioned to prevent said limiter from sliding beyond said second position.

47. An assembly as set forth in claim 46, further comprising a catch engageable when said shield is returned to said first position thereby preventing said shield from moving from said first position to said second position subsequent to administering the intradermal injection.

48. An assembly as set forth in claim 40, further comprising a plug comprising a resilient material applied over said forward tip of said needle cannula thereby sealing said prefillable container at said needle cannula.

49. An assembly as set forth in claim 1, further comprising a prefillable container.

50. An assembly as set forth in claim 12, further comprising a prefillable container.

51. An assembly as set forth in claim 29, further comprising a prefillable container.

52. An assembly as set forth in claim 40, further comprising a prefillable container.
